(19) United States
(12) Patent Application Publication
(10) Pub. No.: US 2025/0129109 A1
(43) Pub. Date: Apr. 24, 2025

(54) SILVER COMPLEX FLUORIDE {BIS (1,3,5-TRIAZA-7-PHOSPHAADAMANTANE) SILVER FLUORIDE} - A NEW THERAPEUTIC AGENT FOR CARIES MANAGEMENT

(71) Applicant: THE UNIVERSITY OF HONG KONG, Hong Kong (CN)

(72) Inventors: Chun Hung CHU, Hong Kong (CN); Ying Kei LUNG, Hong Kong (CN)

(21) Appl. No.: 18/682,313

(22) PCT Filed: Aug. 12, 2022
(86) PCT No.: PCT/CN2022/112047

§ 371 Date: Feb. 8, 2024

Related U.S. Application Data
(60) Provisional application No. 63/260,255, filed on Aug. 13, 2021.

(51) Int. Cl. C07F 9/6584; A61K 8/19; A61K 8/21; A61K 9/00; A61K 33/16; A61K 33/38; A61K 47/10; A61Q 11/00

(52) U.S. Cl. CPC C07F 9/6584; A61K 8/19; A61K 8/21; A61K 9/0056; A61K 33/16; A61K 33/38; A61K 47/10; A61Q 11/00; A61K 2800/58

(57) ABSTRACT

An agent for an arrest of dental caries is a silver complex fluoride (SCF) of the structure: [Ag(1,3,5-triaza-7-phosphaadamantane)₂]F, (Ag(PTA)₂F). The agent effectively arrests dental caries and promotes remineralization of carious lesions. The agent results in minimal staining of teeth and tissue inflammation. The therapeutic agent can be applied selectively to a site of caries or other damaged tissue in the oral cavity.

### FIELD OF THE INVENTION

The present invention relates to a therapeutic agent for caries management, more particularly, a silver complex fluoride agent.

### BACKGROUND OF THE INVENTION

The “fight against cavities” is a common slogan for dentifrices. Fluoride ion from sodium fluoride or other sources of fluoride in dentifrices or rinses have been marketed extensively for many decades to promote resistance to caries through the remineralization and strengthening of enamel and to provide a limited antibacterial effect by fluoride ion. Those that “whiten” or otherwise provide a desirable cosmetic effect are extensively marketed for that perceived need. In recent decades, the use of concentrated antibiotic diamine complexed silver fluorides (SDF) s solutions and sealants have been available for treatment of caries and other oral inflammations. SDF contains Ag⁺ and F⁻ ions. The silver ion exhibits strong antimicrobial activity against a broad spectrum of bacteria. The caries lesion can be re-mineralized by forming a fluoride substituted hydroxyapatite from fluoride ions. The disadvantage of these SDF solutions is the propensity to form “black” silver that permanently stain the teeth, primarily the softer, non-enamel exposed surfaces and gums that effectively stop caries but result in very pronounced discoloration of teeth and gums.

More recently, water-soluble metal phosphine complexes, e.g. Ru, Pt, Au, and Ag have been investigated for their antitumor and antimicrobial activity. As described in some references: Dos Santos E R, et al., New ruthenium (II)/phosphines/diimines complexes: Promising antitumor (human breast cancer) and Mycobacterium tuberculosis fighting agents. Polyhedron 2013; 51:292-297; Zivkovic M D, et al., A new class of platinum (II) complexes with the phosphine ligand pta which show potent anticancer activity. Inorg. Chem. Front., 2018, 5, 39-53; Liu J J, et al., In vitro antitumor and hepatotoxicity profiles of Au(I) and Ag(I) bidentate pyridyl phosphine complexes and relationships to cellular uptake. Journal of Inorganic Biochemistry 2008; 102:303-310; Santini C, et al., In vitro antitumour activity of water soluble Cu(I), Ag(I) and Au(I) complexes supported by hydrophilic alkyl phosphine ligands. Journal of Inorganic Biochemistry. 2011; 105:232-240; Elie B T, et al., Water-Soluble (Phosphane) gold (I) Complexes-Applications as Recyclable Catalysts in a Three-Component Coupling Reaction and as Antimicrobial and Anticancer Agents. Eur. J. Inorg. Chem. 2009, 3421-3430; Pettinari C, et al., Synthesis, Antimicrobial and Antiproliferative Activity of Novel Silver(I) Tris(pyrazolyl)methanesulfonate and 1,3,5-Triaza-7-phosphadamantane Complexes. Inorganic Chemistry 2011, 50, 11173-11183.R.

These complexes include non-nucleophilic anions such as PF₆ and BF₄. Though effective against tumors, malarial and other microbes. Their use of water-soluble phosphines has not been examined as anticaries agents. Phosphine complexed metal fluorides have not been demonstrated for oral remedies.

### BRIEF SUMMARY OF THE INVENTION

A silver complex fluoride (SCF) has a silver cation complexed by a pair of water-soluble phosphine ligands, which can be the same or different, and a fluoride anion, where each of the phosphine ligands includes a phosphorous that bonds to the silver cation and including two or more hydrogen bond acceptors to assure that the SCF is water soluble. The phosphine ligand can be 1,3,5-triaza-7-phosphaadamantane (PTA) where the SCF is [Ag(1,3,5-triaza-7-phosphaadamantane)₂]F, Ag(PTA)₂F.

The SCF can be formulated as the active ingredient of a therapeutic agent where the active SCF is dissolved in water or an aqueous solution that includes other components. The SCF can be [Ag(1,3,5-triaza-7-phosphaadamantane)₂]F, Ag(PTA)₂F. Other components can be included to buffer, thin, thicken, or act as a barrier for the aqueous solution to control the mode of application or to augment the mode of interaction with a surface treated within the oral cavity. Aqueous soluble buffers, low viscosity solvents, such as solvent mixture of ethanol and water, and/or thickening agents can be included. Due to the low solubility of SCF in ethanol, the volume ratio of water to ethanol in the solvent mixture is greater than 1. The aqueous solution of the SCF can be suspended or emulsified in an oil phase.

The SCF can be prepared by combining the water-soluble phosphine ligand with silver fluoride. The silver fluoride in an aqueous solution can be combined with the water-soluble phosphine ligand such that there is little or no excess phosphine ligand in the resulting aqueous solution. The isolation of the SCF is not required for its use as a therapeutic agent: it is applied directly as a solution.

A method of treating dental caries or other ailments in the oral cavity involves the use of the therapeutic agent having the SCF in aqueous solution by universally or selectively applying the therapeutic agent to carious sites within the mouth. The treatment can be by rinsing the entire mouth or by spraying dropping, brushing, or injecting the therapeutic agent to a selected volume within the oral cavity.

### BRIEF DESCRIPTION OF THE DRAWINGS

FIG. 1 shows a mass spectrum of a silver complex fluoride (SCF): [Ag(1,3,5-triaza-7-phosphaadamantane)₂]F, Ag(PTA)₂F.

FIG. 2 shows the staining test results of tooth slices after one week in: 38% silver diamine fluoride (FIG. 2A), 100,000 ppm silver complex fluoride (FIG. 2B), and deionized water (FIG. 2C).

FIG. 3 shows the cell cytotoxic effects of IC50 (silver concentration) for silver complex fluoride (SCF) and silver diamine fluoride (SDF).

### DETAILED DISCLOSURE OF THE INVENTION

In one embodiment, a silver complex fluoride (SCF) is prepared where silver cation is complexed by two water-soluble phosphine ligands in the presence of a fluoride anion. The phosphine ligand can be any ligand that provides a plurality of hydrogen bond acceptors to promote solubility in water and a phosphorous site for strong ligation of silver cation. An exemplary SCF has the structure:

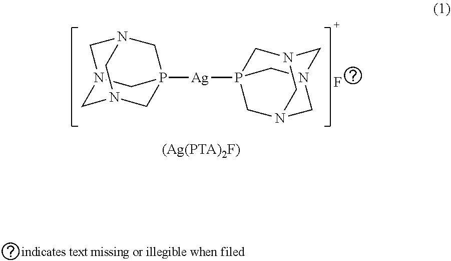

where a pair of 1,3,5-triaza-7-phosphaadamantane (PTA) ligands provide six amine nitrogen hydrogen bond acceptors and two phosphine ligation sites to the Ag ion. Ag(PTA)₂F is stable and highly water soluble while resisting formation of other silver compounds that are black or highly colored such as silver (I) oxide. The silver complex fluorides are more light-stable towards photodecomposition than silver fluoride and many other silver salts. The silver complex fluorides are effective at killing microbes.

The SCF does not require a single phosphine ligand but can contain two different phosphine ligands, each of which having a plurality of hydrogen bond acceptors. The hydrogen bond acceptor can be an amine nitrogen or any other neutral hydrogen bond acceptor. The phosphine ligands can include two, three, or more hydrogen bond acceptors. The hydrogen bond acceptors are selected to have a lesser affinity of ligating a silver cation than does the phosphine of the ligand.

Another embodiment is directed to a therapeutic agent for the treatment of dental caries that includes at least one SCF in an aqueous formulation. The SCF comprising therapeutic agent arrests carious lesions and promotes remineralization of damaged enamel/dentin hard tissue via the formation of fluorapatite. The quantity of the SCF in the therapeutic agent is about five percent to about fifty percent. By comparison, a 3.8% of silver diamine fluoride solution (Saforide R C; 3.8% SDF; Bee Brand Medico Dental) is used for root canal disinfection during endodontic treatment (See Ko A K, et al. Effect of silver diammine fluoride application on dentin bonding performance. Dental Materials Journal 2020; 39:407-414; and, Hiraishi N, et al., Antimicrobial efficacy of 3.8% silver diamine fluoride and its effect on root dentin. Journal of Endodontics 2010; 36:1026-1029).

In the invention, the therapeutic agent does not require an excess of ligand to assure stability of the SCF. Therefore, the therapeutic agent has an SCF with little, less than about five percent, free (unbound) phosphine ligand. The therapeutic agent can be buffered. The SCF can be included in a formulation to coat teeth. The therapeutic agent is effective for gingivitis and does not induce tissue inflammation, such as gingival erythema, gingival inflammation, and mucosal burns that is common to commercial silver diamine fluoride (SDF) solutions used to arrest dental caries.

The therapeutic agent can include a thickening agent based on a natural polysaccharide, polypeptide, or any other natural water-soluble thickening agent. The thickening agent can include a synthetic polyether, polyamide, or other biocompatible non-toxic thickening agent. The therapeutic agent can include an oil where the aqueous solution is suspended or emulsified in a water-in-oil emulsion. Any emulsifying agent does not contain a phosphine or other functionality that competitively complexes with silver to substitute phosphine ligand such that the SCF is stable in the aqueous phase. The oil can be a natural biologically sourced or derived oil or a synthetic oil. The oil can provide a vehicle for the aqueous SCF solution. The oil of the emulsion may include a single functionality, such as an olefin or multiple complementary functionalities that can be employed for cross-linking. As needed, a cross-linking agent can be used to form a matrix from at least a portion of the oil phase to encapsulate the SCF comprising aqueous phase within a stabilize coating on a tooth or other oral surface. Cross-linking can be by a reaction between self- or cross-complimentary functionalities that slowly react after mixing shortly before application of the therapeutic agent. Cross-linking can be initiated or promoted by heating or irradiation with electromagnetic radiation, such as near-IR, visible, or near-UV radiation in a manner where photoreduction of the silver is minimal. The cross-linking should not be performed at much elevated temperature for stability concern of the SCF. Suitably, the cross-linking can be initiated or promoted at the temperature of 50˜70° C.

Another embodiment is directed to a method of treating caries where a therapeutic agent is applied to surfaces in the oral cavity. Application involves delivering an aqueous based liquid formulation that includes a SCF as a rinsing solution for delivery over the entire oral cavity or by delivery from a dropper, brush, sprayer, syringe, or any other device that allows selective placement of the therapeutic agent to a specific surface within the oral cavity. The delivery can be directly to an identified cavity or a surface that displays inflammation or other indication of microbial induced degradation within the mouth. The treatment can require a single application, or a plurality of applications spaced over time as determined by a dental professional.

The treatment can employ a therapeutic agent formulation with a desired viscosity, that ranges from a solution of the SCF in otherwise pure water or even less viscous mixtures with organic solvents, such as ethanol+water, acetone+water, to highly viscous solutions formed using natural or artificial non-toxic thickeners. Due to the low solubility of AgF (and SCF) in ethanol/acetone, the volume ratio of water to ethanol/acetone in the corresponding mixture is greater than 1. The formulation can be a paste that can be pressed or otherwise molded around a surface in the mouth by a dental technician. The aqueous therapeutic agent can be included in a water-in-oil emulsion where the continuous oil phase allows the diffusion of the aqueous phase comprising the SCF to a hydrophilic oral surface while isolating the aqueous phase from the natural aqueous environment to suppress treatment of other portions of the oral cavity for a period determined. As such, the efficiency of the therapeutic agent in targeted portions is maximized while minimizing undesirable effects in other portions. The oil phase can be gelled about the treated surface to enhance isolation of the SCF to the desired volume in the oral cavity.

In the invention, the treatment applies a very small volume of silver complex fluoride to carious lesions, e.g., in the form of SCF solution by a small brush. The F-ions provided by SCF will form insoluble fluorapatite or calcium fluoride on dentin or enamel surfaces, which will minimize the ingestion of F-ions into the body and thus reduces the Fions toxicity. Therefore, it is safe to use silver complex fluoride to manage dental caries.

### Materials and Methods

### Preparation of a SCF and Aqueous Therapeutic Agent

A SCF can be prepared by using silver fluoride AgF and PTA at a molar ratio of from 1:1.1 to 1:4.1, 1:2.1, or 1:4.1.

In an example, the synthesis of [Ag(1,3,5-triaza-7-phosphaadamantane)₂]F, Ag(PTA)₂F was carried out in aqueous solution in the absence of light. The SCF was prepared with a 1:4.1 molar ratio of silver fluoride (AgF) to PTA. An aqueous solution of silver fluoride (0.05 g, 0.4 mmol) in 16 mL deionized water was prepared and mixed with PTA (0.26 g, 16.6 mmol) at room temperature with stirring for three hours to form the Ag(PTA)₂F comprising therapeutic agent according to mass spectroscopy rather than Ag(PTA)₄F from the stoichiometry employed.

In another example, the synthesis of [Ag(1,3,5-triaza-7-phosphaadamantane)₂]F, Ag(PTA)₂F (1) was carried out in aqueous solution in the absence of light. The SCF was prepared with a 1:2.1 molar ratio of silver fluoride (AgF) to PTA. An aqueous solution of silver fluoride (0.5 g, 3.94 mmol) in 5 mL deionized water was prepared and mixed with PTA (1.30 g, 8.27 mmol) at room temperature with stirring for three hours to form the Ag(PTA)₂F comprising therapeutic agent.

To prepare high concentration of SCF (≥100,000 ppm), heating may be required in order to dissolve the ligand, PTA. In an example, the synthesis of [Ag(1,3,5-triaza-7-phosphaadamantane)₂]F, Ag(PTA)₂F (1) was carried out in aqueous solution in the absence of light. The SCF was prepared with a 1:2.5 molar ratio of silver fluoride (AgF) to PTA. The PTA (0.93 g, 5.92 mmol) in 3 mL deionized water was heated to 50° C. When all PTA was dissolved, the solution was cooled down to room temperature. An aqueous solution of silver fluoride (0.3 g, 2.36 mmol) was prepared and mixed with the PTA solution at room temperature for three hours to form the Ag(PTA)₂F comprising therapeutic agent.

### Characterization of Ag(PTA)₂F

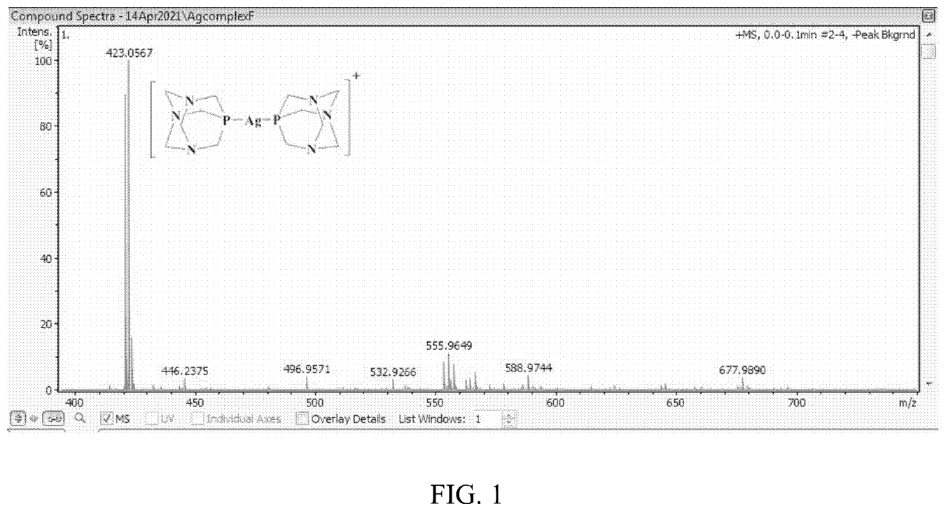

The Ag(PTA)₂F was characterized by mass spectroscopy, as shown in FIG. 1, where the Ag(PTA)₂⁺ ion displays the 421 and 423 m/z signals that indicate the natural isotopic distribution of 107 and 109 silver.

### Staining Test

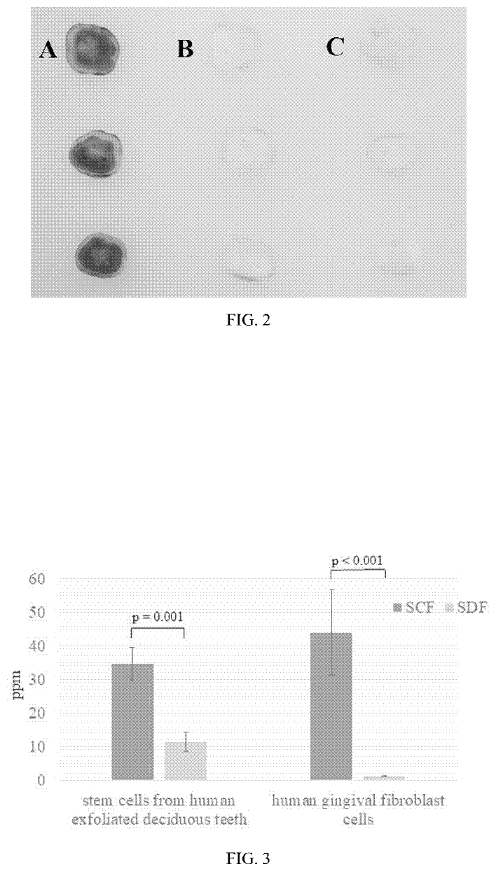

The staining test was performed by immersing tooth slices for one week in the following: 38% silver diamine fluoride, 100,000 ppm silver complex fluoride and deionized water. As shown in FIG. 2, silver diamine fluoride stained the demineralised tooth slices black, while little staining was observed on demineralised enamel and dentine on the tooth slices by silver complex fluoride solution for one week.

### Cell Cytotoxic Test

The cell cytotoxic test was performed for the silver complex fluoride (SCF) and the silver diamine fluoride (SDF) using human gingival fibroblast cells (HGF-1) and stem cells from human exfoliated deciduous teeth (SHED1), with the test results shown in FIG. 3. The IC50 value of SCF and SDF against HGF-1 were 43.98±12.80 ppm and 1.30±0.13 ppm, respectively (p<0.001); whereas the IC50 value of SCF and SDF against SHED1 were 34.54±5.09 ppm and 11.38±2.89 ppm respectively (p=0.001). Thus, it is demonstrated that the cell cytotoxicity of SCF to HGF-1 and SHED1 cells was lower than SDF.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

1. A silver complex fluoride (SCF), comprising:
a silver cation;
two water-soluble phosphine ligands where each of the phosphine ligands comprises a phosphorous ligated to the silver cation and a plurality of hydrogen bond acceptors; and
a fluoride anion, wherein the SCF is water soluble.

2. The SCF according to claim 1, wherein each of the phosphine ligands is identical to the other.

3. The SCF according to claim 1, wherein the water-soluble phosphine ligand is 1,3,5-triaza-7-phosphaadamantane (PTA).

4. The SCF according to claim 1, wherein the SCF is [Ag(1,3,5-triaza-7-phosphaadamantane)₂]F, Ag(PTA)₂F.

5. A therapeutic agent, comprising:
at least one silver complex fluoride (SCF) according to claim 1; and
a solvent, wherein the SCF resides in an aqueous solution.

6. The therapeutic agent according to claim 5, wherein the SCF is [Ag(1,3,5-triaza-7-phosphaadamantane)₂]F, Ag(PTA)₂F.

7. The therapeutic agent according to claim 5, wherein the solvent is water.

8. The therapeutic agent according to claim 5, wherein the aqueous solution further comprises a buffer.

9. The therapeutic agent according to claim 5, wherein the aqueous solution further comprises a thickener.

10. The therapeutic agent according to claim 5, wherein the aqueous solution further comprises a second solvent, wherein the second solvent is water miscible.

11. The therapeutic agent according to claim 10, wherein the second solvent is ethanol.

12. The therapeutic agent according to claim 5, further comprising an oil wherein the aqueous solution is suspended or emulsified within the oil.

13. A method of preparing a SCF, comprising combining a pair of water-soluble phosphine ligands with silver fluoride, wherein the phosphine ligand comprises a phosphorous atom and a plurality of hydrogen bond acceptors.

14. The method according to claim 13, wherein the phosphine ligand is 1,3,5-triaza-7-phosphaadamantane (PTA).

15. The method according to claim 13, wherein the phosphine ligand is in an aqueous solution.

16. The method according to claim 13, wherein the silver fluoride is in an aqueous solution.

17. A method of treating dental caries, comprising:
providing a therapeutic agent according to claim 5; and
applying the therapeutic agent to at least one surface within an oral cavity.

18. The method according to claim 17, wherein the therapeutic agent further comprises a second solvent, a thickener, a buffer, and/or an oil.

19. The method according to claim 17, wherein applying comprises rinsing, spraying dropping, brushing, injecting, or any combination thereof.

20. The method according to claim 17, further comprising cross-linking wherein the therapeutic agent further comprises an oil comprising at least one component with a cross-linking functionality and optionally comprising a cross-linking agent.
